(19) United States
(12) Patent Application Publication
(10) Pub. No.: US 2007/0225544 A1
(43) Pub. Date: Sep. 27, 2007

(54) APPARATUSES AND TECHNIQUES FOR BIOACTIVE DRUG DELIVERY IN THE PROSTATE GLAND

(76) Inventors: Waseet VANCE, New York, NY (US); Shravan Kambam, New York, NY (US)

Correspondence Address: REED SMITH LLP, P.O. BOX 488, PITTSBURGH, PA, 15230-0488, US

(21) Appl. No.: 11/676,805

(22) Filed: Feb. 20, 2007

Related U.S. Application Data
(60) Provisional application No. 60/774,495, filed on Feb. 17, 2006.
(60) Provisional application No. 60/795,339, filed on Apr. 26, 2006.
(60) Provisional application No. 60/795,338, filed on Apr. 26, 2006.

(51) Int. Cl. A61N 5/00; A61M 36/00

(52) U.S. Cl. 600/8; 600/3

(57) ABSTRACT

Methods and apparatuses for the delivery of bioactive substances into the prostate. The present invention encompasses the release of bioactive substances into the prostate of a patient. Preferably, the present invention employs either a sustained-release or depot formulation of the bioactive substance to release the substance over an extended period of time. In particularly-preferred embodiments, the apparatuses of the present invention release of anti-inflammatory agents into the prostate. The anti-inflammatory agents preferably reduce the inflammation that is associated with brachytherapy and other conditions of the prostate. In particular, corticosteroid anti-inflammatory agents are employed in the context of the present invention.

### CROSS-REFERENCE TO RELATED APPLICATION

This application claims the benefit under 35 U.S.C. §119(e) of the earlier filing date of U.S. Provisional Application Ser. No. 60/774,495 filed on Feb. 17, 2006; 60/795,339 filed on Apr. 26, 2006; and 60/795,338 filed on Apr. 26, 2006.

### BACKGROUND OF THE INVENTION

1. Field of the Invention

The present invention relates generally to devices, drugs, and techniques for the effective and safe delivery of drugs into the prostate. More particularly, the present invention relates to novel methods and apparatuses for the administration of anti-inflammatory agents directly into the prostate gland using a drug eluting polymer.

2. Background

Cancer affects 40% of males and 30% of females in the United States and has now surpassed heart disease as the number one cause of death under age of 85. Excluding skin cancer, prostate cancer is the number one male cancer and the second-most common cancer overall (behind lung cancer) in the United States. 1 in 6 males will be diagnosed with prostate cancer. However, autopsy specimens predict that almost 4 of 6 males (greater than 60% of males) harbor the disease. Prostate cancer has been a disease of older men. However, the American Cancer Society estimates that during 2006 about 234,460 new cases of prostate cancer will be diagnosed in the United States—many of these under the age of 60. As the U.S. population over fifty rises in the next two decades, the number of cases of prostate cancer will rise dramatically. In order to reduce the incidence, prostate cancer prevention trials are underway which utilize vitamins or systemic (e.g., finasteride) manipulation of testosterone derivative levels. However, no major convincing data has led to current widespread use of prevention techniques in the general public or in high-risk individuals. The primary advance in prostate cancer treatment in the past 20 years has been early identification of cancers with prostate-specific antigen (PSA) screening.

Treatment for prostate cancer is based on an estimate of the extent of disease. Clinical stage of the cancer, PSA level, the histological grade and extensiveness of the tumor as seen on biopsy are risk factors which allow physicians to estimate the likelihood of disease outside the gland. From this analysis, patients are placed into risk groups (low, intermediate and high) which helps clarify the management options.

Because of PSA screening, more than 90% of all prostate cancers are found in the low risk, local stage (i.e., confined to the prostate gland or peri-prostatic tissue). The treatment modalities currently and commonly employed for low risk disease include radical prostate surgery and radiation therapy (Brachytherapy and External beam).

For more advanced disease (intermediate and high risk) combinations of therapies are often employed. Patients in these categories are at greater risk to have disease beyond the prostate and are therefore treated after surgery, before implantation or after localized external beam radiation to wider external beam fields which may include the pelvic lymph nodes. Hormonal therapy generally is utilized for high risk prostate cancer and for patients with known metastasis. Systemic chemotherapy is generally reserved for treating metastatic disease.

Long term results for surgical, brachytherapy and external beam are quite similar for early stage disease. The primary advantage of brachytherapy or external beam radiation therapy over surgery includes decreased morbidity, as defined by reduced urinary complications and decreased impotence rates. For that reason, many low-risk, localized prostate cancer patients opt for radiation over surgery. As currently and commonly used, radiation therapy can be delivered to the prostate gland by use of external beam radiation therapy (x-rays delivered from outside the body, generated by a linear accelerator), interstitial brachytherapy (the placement of radioactive “seeds” directly into the prostate gland), or a combination of external beam radiation and brachytherapy. The most commonly used radioactive isotopes used in prostate brachytherapy are iodine-125 and palladium-103.

Most localized prostate cancers can be managed and cured by prostate seed implant, external beam radiation, or the combination of both modalities. Prostate Brachytherapy (seed implantation) is generally a simple one hour outpatient surgical procedure involving the insertion of usually 20-30 hollow needles (catheters) through the perineal skin and into the prostate gland. Rectal ultrasound guidance or other imaging modalities (e.g., MRI guidance), as shown in FIG. 1 may be used to guide the needles.

Placement of seeds requires carefully planning. The procedure may be pre-planned prior to the procedure or intraoperatively, during the procedure. Both techniques require a detailed rectal ultrasound mapping outlining the shape and size of the prostate. Using such volumetric data of the gland and knowledge of the required dose, a radiation physicist determines the number of seeds and location of seed placement within the prostate gland required to deliver the prescribed dose. The prostate gland and a small amount of surrounding tissue are included in this treatment volume. After the implant, a CT scan is performed and calculations made to determine the actual dose and quality of the implant.

During the procedure, radioactive seeds are introduced into the prostate via needles. These needles may be preloaded with seeds or alternatively loaded after the needle is placed. With the Mick afterloading system, the radioactive seeds are released individually into the prostate gland after a trocar and sleeve are placed into the target. Illustrations of the Mick afterloading system, including a seed cartridge and the needles through which seeds are delivered are shown in FIG. 2.

With either preloaded or the Mick System, the needle and subsequent seed placement follows a pre or intraoperative plan. The needle and seed placement is done under ultrasound guidance, such that relatively equal spacing and distribution of seeds is achieved and the plan requirements met. The goal is a relatively a homogenous radiation dose distribution covering the entire prostate gland. Preloaded needle systems have an advantage over the Mick system as they allow for multiple, connected seeds to be placed into the prostate gland with each needle. Spatial separation of seeds may be achieved by the use of intervening non-radioactive material (known as “spacers”) which are either simply placed in a loose single-file pattern in between the radioactive seeds or physically fixed to each other and the seeds by a surrounding polymeric material (e.g., “Rapid Strand” system). Individual seeds are shown in FIG. 3.

As discussed above, iodine-125 is the radioactive isotope most commonly used in radioactive seeds and has a half-life of 60 days and an effective life of 300 days. The second most commonly used isotope is palladium-103 which has a half life of 17 days. The size and shape of both the iodine-125 and palladium-103 seeds are identical. Iodine-125 and Pd 103 emit low energy beta and gamma radiation which penetrates a short distance, resulting in rapid dose degradation such that the emitted radiation can only reach a few millimeters in maximal path length—enough to cover the prostate gland, but not significantly beyond into surrounding tissues. Usually, between 60 and 120 seeds are inserted into a cancerous gland via 20-30 needles. Other isotopes such as Cesium 131 are currently being explored. The distribution of radioactive seeds as currently and commonly used in prostate brachytherapy following the surgical procedure may be found in FIG. 5.

Most preloaded needles today incorporate connected seed technology. However, some centers still use older methods and load the needles with independent or free seeds that are not physically connected. These seeds may migrate short distances in the prostate and further if placed into the veins surrounding the gland. Neither preoperative nor intraoperative planning techniques have been proved superior in reducing this tendency. However, the use of connected seeds has reduced this substantially and improved overall dosimetry. With either free or connected seeds, multiple seeds can be placed incorrectly or migrate slightly resulting in an inhomogeneous dose distribution within the prostate gland. This can potentially result in underdosing cancer cells or alternatively overdosing normal tissue.

Underdosed regions may result in recurrent cancer spread (metastasis), and patient death. An underdosed region of prostate seed implant is typically recognized with post implant CT dosimetry and is retreated with reimplantation of additional seeds or, rarely with an additional course of external beam radiation. Overdosing may also occur. If the seeds bunch too close together and overdose critical structures such as the rectum, bladder neck, urethra, or peri-prostatic neurovascular bundles, unwanted morbidities such as radiation proctitis or tenesmus, radiation urethritis (causing dysuria and/or hematuria), bladder neck spasticity (causing urgency, incontinence, etc.), prostatitis, or impotence may result.

As noted, various techniques are used in prostate seed implants to ensure homogeneous and adequate seed number and placement location/distribution. To reduce the risk of seed migration (causing inhomogeneous, underdosed or overdosed areas within the cancerous prostate), as mentioned above, a system of inserting an entire group of connected radioactive seeds as a “needle” unit has been introduced. These “seed links or strands” which are commercially available consists of ˜2-6 radioactive seeds each precisely spaced apart but connected by an inert biodegradable/absorbable non-radioactive material. The connected seed string thus formed creates an alternating seed, “spacer”, seed, etc. “string of pearl” pattern that ensures the correct physical separation, spatial positioning and stability of the radioactive seeds within the prostate gland for homogenous radiation dose delivery. The “spacers” are biologically inert, bio-absorbable and dissolve with time, and pose no harm or damage to the patient.

Inserting the needle with preloaded, connected seeds shortens the overall surgical time and allows for more reliable positioning of seeds, since the linear “string of pearls” arrangement of “spacers” between consecutive radioactive seeds limits seed migration, and thus improves accuracy of planning and homogeneity of dose delivery to the prostate. Pre-loaded needle techniques are typically employed with pre-planned implants because flexibility of seed adjustment with pre-loaded needles during the brachytherapy procedure.

As discussed above, various treatment planning systems are employed prior to the implant to guide the physician in the placement of the radioactive seeds into the prostate gland with precise spatial distribution to allow for the planned radiation dose delivery to the target volume of interest. Treatment planning systems are computer-based programs typically based on ultrasound imaging of the prostate gland. The program generates a pre-plan or intra-op plan for radioactive seed placement to deliver the appropriate dose the prostate target volume by integrating radioactive seed strength, radiation dose falloff of individual seeds, and relating the cumulative dose to the target volume. The total dose “topographically” is based on the dose “interaction” of all the implanted seeds within the prostate gland.

While most prostate brachytherapy procedures such as those mentioned above involve the permanent placement of radioactive seeds into the prostate gland for radiation delivery over a substantial period of time (termed low dose rate brachytherapy or LDR), a minority of prostate cancer brachytherapy procedures involve what is termed as high dose rate brachytherapy (or HDR). HDR involves the placement of hollow plastic catheters (typically introduced inside hollow stainless steel needles) into the prostate gland, followed by the “afterloading” of a radiation source (e.g., Ir-192) into these catheters for a defined period of time. After radiation is delivered by the HDR source from variable positions within these catheters to the prostate gland as per the computer-generated plan for appropriate radiation dose delivery, the radiation source is retracted from the prostate gland and the catheters are removed from the prostate gland.

Both external beam radiation and prostate seed implant (i.e., brachytherapy) techniques cause inflammation of the prostate gland secondary to the intrinsically damaging effects of radiation on tissue. However initial swelling of the gland is due to the brachytherapy needle insertion into the prostate gland. Relevant to the present invention, inflammation, swelling, and subsequent related symptoms and long term effects from prostate seed implant are due to both the surgical trauma and the radiation effects of the seeds. The literature states that the surgical swelling and enlargement of the gland has a half life of approximately 30 days.

Seed implantation is designed for a normal/non-enlarged prostate. The placement and total number of radioactive seeds is designed to achieve proper dose distribution within the pre-implant gland volume. The surgical swelling immediately after the implant can result in the seeds separating a small degree, affecting the overall dose distribution. The surgically-induced enlargement of the prostate therefore, in most patients results in the seeds being further apart from each other until this swelling resolves. The planning of seed placement cannot predict accurately such post-implant prostate volume enlargement. Since the degree of enlargement of the prostate gland cannot be predicted and can be quite variable, no effort is made to “guess” on the volume changes. Dose prescriptions have taken into account this enlargement but since not all glands swell proportionately the same amount, it would be preferable and ideal to devise a method to minimize the gland enlargement particularly during the first 30 day when the majority of enlargement occurs. While local cancer control rates have not yet been affected by this enlargement, the potential for high dose and low regions remains and these high dose regions likely have significant effect on the acute and chronic side effects.

The half life of the radioactive seeds (60 days for iodine-125 and 17 days for palladium-103). During this time, the initial dose prescribed to a pre implant volume is now affected by a larger post implant volume. Since this dose had been planned prior to the enlargement of the gland, by the time the enlarged prostate gland returns back to its normal pre-implant size, the decay of the radioactive seeds may result in diminished dose to planned region. Thus, enlargement of the prostate gland secondary to surgical trauma may result in a decrease in radiation dose to the initially-planned prostate volume. As current techniques are quite successful in controlling disease locally, it suggests that more radiation is being given than is necessary. It is anticipated that if this swelling effect can be reduced, there is a potential for reducing the prescription dose and therefore long term complications.

A more acute impact of the surgical prostate gland enlargement and swelling after seed implant is the acute patient morbidity relating to urinary difficulty (obstruction) and discomfort. All patients experience varying degrees of urination related morbidity related to the procedure. Most patients experience some pain on initial urinations after the procedure. Approximately 5-10% of patients require a temporary catheter and, in rare circumstances, a catheter for a period of many months. The immediate effects are related to the immediate surgical induced enlargement of the gland and, in the ensuing months, effects by the radiation induced swelling within the gland. Patients may experience the radiation-induced swelling as a slowing of the urinary stream, slight pain, urgency, or frequency. These symptoms are caused by compression of the urethra (the central canal through which urine flows from the bladder through the prostate and through the penis). Compression of the urethra by internal prostate swelling can cause, in more extreme situations, complete urinary obstruction. If medication cannot keep the passage adequately patent after seed implantation, then the patient will require either Foley catheterization, daily self-catheterization, or suprapubic diverting cystostomy. Persistent swelling and obstruction may ultimately result in eventual surgical intervention (e.g., transurethral resection of a portion of urethra and prostate to allow for urine flow). While these obstructive and irritative symptoms may occur as the primary morbidities following prostate seed implant, lesser but relevant problems include rectal morbidities (proctitis, tenesmus, etc.) and sexual dysfunction (impotence) related to the implant procedure. Attempts to decrease these effects seem warranted.

Prostatitis is a common and non malignant ailment in men. This inflammatory process can be the result of both bacterial and non bacterial causes. While many of the causes are treatable with systemic agents or are self limiting, many men have persistent symptoms that are not amenable to standard treatment.

Anti-inflammatory medications are used to reduce swelling and associated symptoms in multiple conditions. They can generally be broken down into two major classes by their mechanism of action. Non-steroidal anti-inflammatory drugs (NSAIDS) work by inhibiting COX-1 and/or COX-2 receptors at the cellular level to mediate the inflammatory response. In contrast, corticosteroids are a specific class of anti-inflammatory “steroidal” medications that are related to cortisone. In contrast to NSAIDS, members of this steroid class reduce inflammation much more powerfully. These agents work at the cellular level to suppress fibroblasts, DNA, RNA, and protein synthesis. As opposed to NSAIDS whose clinical use is typically related to systemic administration (via oral or intramuscular delivery), corticosteroids can be used for either systemic or localized effects—whether taken by mouth, inhaled, applied to the skin, given intravenously, or injected directly into the tissues of the body. Examples of corticosteroids include prednisone and prednisolone (given orally), solumedrol (given intravenously), as well as triamcinolone, celestone, depomedrol and others (given by injection into body tissues for local effect).

Essentially, corticosteroids reduce inflammation regardless of the method of administration. Corticosteroid injections can be used to treat the inflammation of small areas of the body (local injections) or they can be used to treat inflammation that is widespread throughout the body (systemic injections). Examples of conditions for which local cortisone injections are used include inflammation of a bursa (bursitis), a tendon (tendonitis), and a joint (arthritis). In the latter form, corticosteroid infiltrations are among the most accepted, most frequently administered, and most successful treatments used by rheumatologists, orthopedic surgeons, and primary care physicians. They stand in sharp contrast to systemic (oral or parenteral) corticosteroids, which are rightfully used with caution because of myriad cumulative side effects. On the other hand, intraarticular and soft tissue corticosteroid infiltrations exert most of their anti-inflammatory action locally, with only a small amount entering into the circulation in the first 24 hours post-injection. This allows long-term local improvement to occur without systemic side effects.

Advantages of local administration of corticosteroidal compounds include the rapid onset of the medication's action, dependability, and minimal side effects. Another distinct benefit of a corticosteroid injection is that the relief of localized inflammation in a particular body area is more rapid and powerful than with traditional anti-inflammatory medications given orally (e.g., NSAIDS such as aspirin or corticosteroids such as prednisone). While the inflammation for which corticosteroids are given can recur, corticosteroid injections can provide months to years of relief when used properly.

Injectable Corticosteroid Preparations

Both soluble and depot corticosteroid preparations exist. Soluble formulations (e.g., hydrocortisone) have little role in infiltration therapy because they diffuse readily from the injected region and exert predominately systemic effects. The depot forms (methylprednisolone acetate, triamcinolone acetonide, triamcinolone hexacetonide) are crystalline, bulky, and relatively impervious to degrading enzymes, so they tend to remain at the injected site for a prolonged period. For example, intraarticular corticosteroids may be seen as free-floating or intracellular crystals several weeks after injection. Various formulations of corticosteroids exist—each with its own potency, onset, and duration of action making this class of drugs a very broad and versatile group with a tremendous range of applications.

Examples of Injectable Corticosteroid Agents
by Relative Potencies, Duration, and Dose

| Agent | Potency | Duration | Dose/site |
|---|---|---|---|
| Hydrocortisone acetate | Low | Short | 10 to 25 mg for |
| (Hydrocortone) | | | soft tissue and |
| | | | small joints |
| | | | 50 mg for large |
| | | | joints |
| Methylprednisolone acetate | Inter- | Inter- | 2 to 10 mg for |
| (Depo-Medrol) or triamcinolone | mediate | mediate | soft tissue and |
| acetonide (Aristocort) | | | small joints |
| | | | 10 to 80 mg for |
| | | | large joints |
| Dexamethasone sodium phosphate | High | Long | 0.5 to 3 mg for |
| (Decadron) | | | soft tissue and |
| | | | small joints |
| | | | 2 to 4 mg for |
| | | | large joints |
| Betamethasone sodium phosphate | High | Long | 1 to 3 mg for |
| and acetate (Celestone Soluspan) | | | soft tissue and |
| | | | small joints |
| | | | 2 to 6 mg for |
| | | | large joints |

In summary, in low-risk localized prostate cancer, prostate seed implant alone is a popular management strategy allowing most patients to be treated with a one-day, outpatient procedure. In contrast to the inconvenience related to 8-9 weeks of external beam radiation therapy or the morbidity of a radical prostatectomy, brachytherapy (seed implantation) has become a standard treatment strategy both for early and more advanced prostate cancer. Brachytherapy in conjunction with external beam radiation is also used in intermediate or high-risk localized prostate cancer. Since most patients with prostate cancer will be alive and free of disease for many years, these patients would greatly benefit from any significant advancement in reducing the side effects of the treatment. Patients with inflammatory conditions of the prostate may also benefit from this treatment. The apparatuses and methods for the local application of a bioactive pharmaceutical compound(s) generally, and specifically corticosteroids, that would be effective in reducing radiation and surgical trauma induced enlargement and swelling of the gland of the prostate gland could potentially provide both substantial reduction of treatment-related morbidity and allow for lower doses of radiation. There has been a long-standing need within the medical community to reduce the local trauma and morbidity associated with brachytherapy and HDR irradiation techniques. For benign conditions this approach may provide relief in situations where simpler techniques are not successful.

### BRIEF DESCRIPTION OF THE DRAWINGS

For the present invention to be clearly understood and readily practiced, the present invention will be described in conjunction with the following figures, wherein like reference characters designate the same or similar elements, which figures are incorporated into and constitute a part of the specification, wherein:

FIG. 1 depicts brachytherapy techniques that employ ultrasound-image guidance;

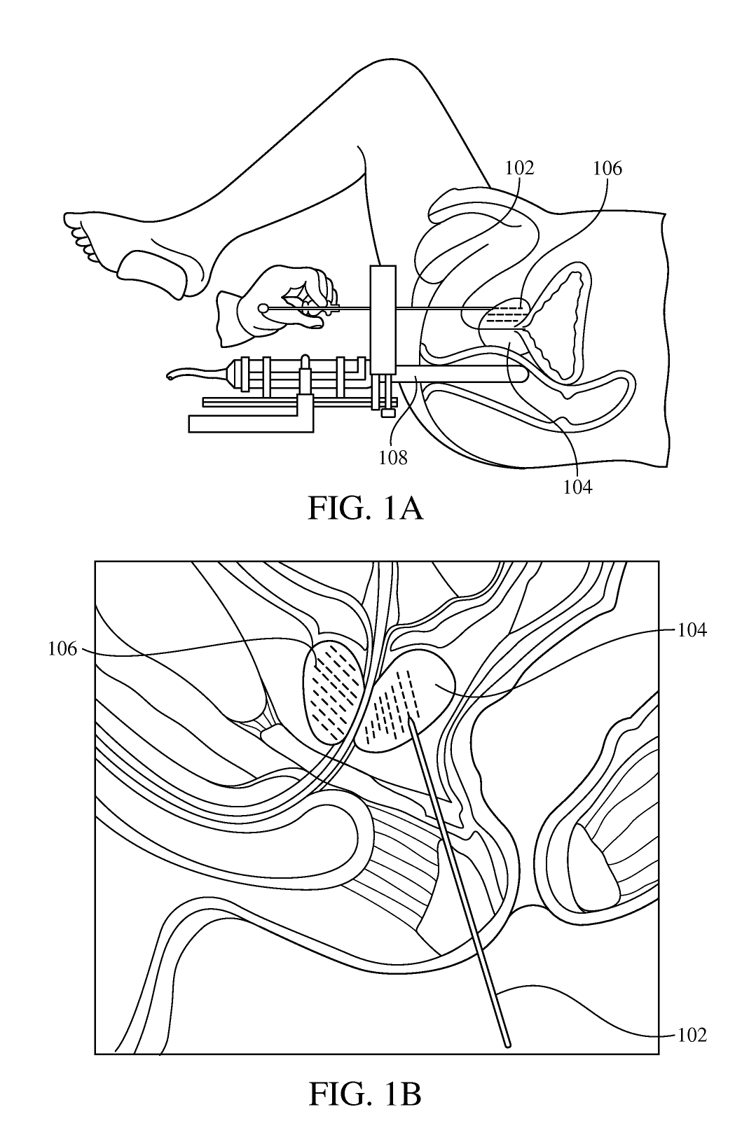

FIG. 2 shows an implementation of the MICK system;

FIG. 3 displays individual radioactive seeds that are employed used in brachytherapy;

FIG. 4 displays the radioactive seeds separated by spacers dispersed within a prostate gland;

FIG. 5 shows the distribution of radioactive seeds following brachytherapy; and

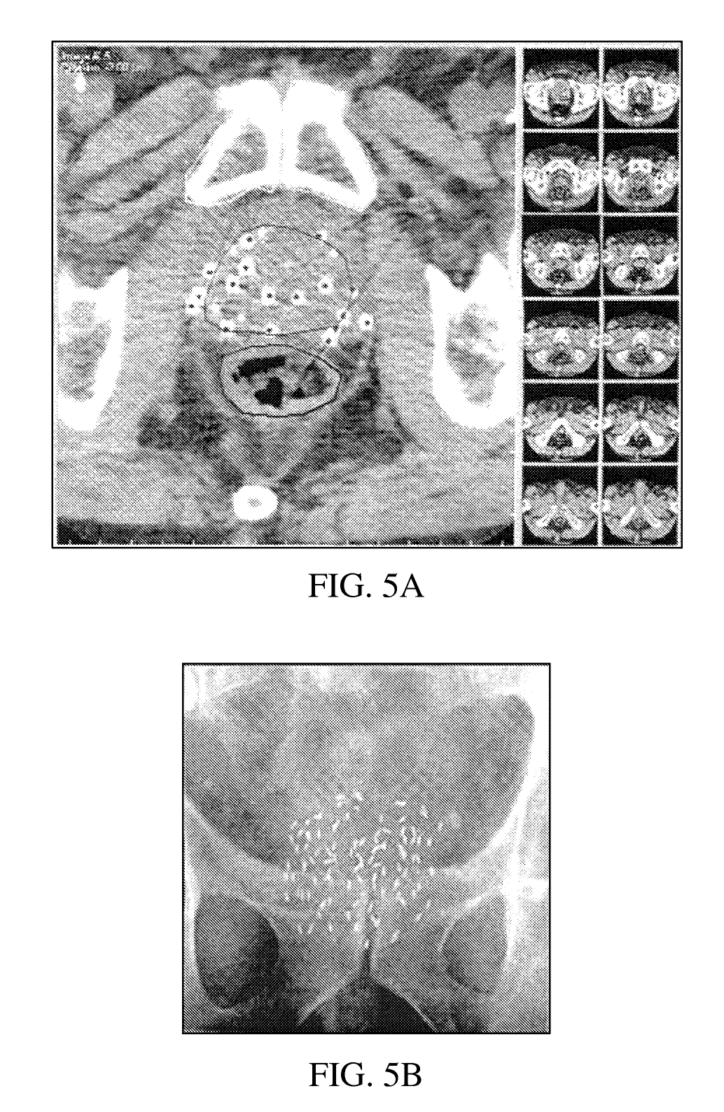

FIG. 6 displays an applicator system of the present invention that is capable of administering radioactive seeds and drug-eluting polymers to a patient.

### SUMMARY OF THE INVENTION

The present invention generally relates to the release of bioactive substance into the prostate of a patient. The patient may be suffering from prostate cancer or an inflammation of the prostate. The bioactive substance may be employed to treat the prostate gland of the patient or to reduce the inflammation of the prostate gland and/or tissues surrounding the prostate gland. In preferred embodiments, the bioactive substance is an anti-inflammatory agent. In particularly-preferred embodiments, the bioactive substance is a steroid inflammatory agent and, more specifically, a corticosteroid.

The bioactive substance may be released into the prostate gland by a variety of mechanisms. For example, the invention may employ a drug-eluting polymer or a depot formulation. The bioactive substance may be incorporated into the drug-eluting polymer, which then releases the bioactive substance over a prolonged period. A diversity of drug-eluting polymers is well-known in the art. The drug-eluting polymer may remain in the prostate gland permanently or be bio-erodable and decay over time.

The apparatuses of the present invention are particularly well suited for use in commonly-employed medical techniques. For example, the present invention may be employed in brachytherapy. The bioactive substance may be incorporated into a drug-eluting polymer which may serve as individual capsules throughout the gland or spacers between the radioactive seeds that are implanted into the prostate during brachytherapy. Alternatively, the drug-eluting polymers of the present invention may coat radioactive seeds or encase a string of radioactive seeds. In cases where the bioactive substance is an anti-inflammatory corticosteroid, the present invention would have the benefit of reducing inflammation of the prostate that routinely results from the surgical trauma and radioactivity exposure that accompanies brachytherapy. In preferred embodiments, the apparatuses of the present invention provide for the release of anti-inflammatory agents over a time period where the prostate is exposed to radioactivity. In addition, the apparatuses and methods of the present invention may be employed in high dose radioactivity treatments of prostate cancer where the prostate is exposed to an external radioactivity beam. In such embodiments, the apparatuses of the present invention may be implanted directly into the prostate to reduce the inflammation associated with exposure to the high energy radioactivity.

### DESCRIPTION OF THE INVENTION

It is to be understood that the figures and descriptions of the present invention have been simplified to illustrate elements that are relevant for a clear understanding of the invention, while eliminating, for purposes of clarity, other elements that may be well known. The detailed description will be provided hereinbelow with reference to the attached drawings.

The present invention generally relates to methods and apparatuses for the administration of bioactive compounds directly into a target tissue. In certain preferred embodiments, the target tissue is the prostate gland. In certain preferred embodiments, the present invention relates to methods and apparatuses that reduce the inflammation of the prostate by administration of anti-inflammatory agents directly into the prostate through a sustained-release drug delivery mechanism. In particularly-preferred embodiments, the present invention encompasses sustained-release formulations employing drug-eluting polymers or other compounds (e.g., ceramics) and bio-polymers (e.g., phosphorylcholine) that are effective for releasing anti-inflammatory corticosteroids over an extended period of time. In other particularly-preferred embodiments, the present invention encompasses sustained-release depot formulations that are effective for releasing anti-inflammatory corticosteroids over an extended period of time.

In presently-preferred embodiments, the target tissue is the prostate. As noted hereinabove, treatments that are employed to treat prostate cancer result in the inflammation the prostate. In particular, the extended placement of radioactive seeds in the prostate (as practiced in LDR brachytherapy) and repeated radiation source exposure (as practiced in HDR brachythreapy) results in inflammation of the prostate and a variety of undesirable side effects. The present invention preferably overcomes these problems of the prior art by administering anti-inflammatory agents into the prostate along with the radioactive seeds. In presently-preferred implementations, the anti-inflammatory agent is a corticosteroid anti-inflammatory agent.

In certain preferred embodiments, the present invention includes apparatuses and methods that provide for the local administration of a pharmaceutical compound with anti-inflammatory properties to achieve enhanced efficacy and decreased morbidity relating to the delivery of radiation therapy (in the form of external beam radiation or brachytherapy) in the treatment of prostate cancer or other diseases of the prostate gland. In presently-preferred embodiments, the anti-inflammatory compound is released from a drug-eluting polymer that is associated with the radioactive seeds that are traditionally employed with brachytherapy. The drug-eluting polymer may coat the seeds, may form the spacers between the seeds, or may be inserted into the prostate at the same time as the seeds.

As detailed more fully hereinbelow, the anti-inflammatory compound may be a steroid or non-steroid, with steroids being preferred. Anti-inflammatory agents delivered locally may improve clinical efficacy of radiation therapy dose delivery by reducing surgical trauma-related or radiation related enlargement of the prostate gland thereby decreasing obstructive and irritative morbidities such as urinary flow obstruction, urethritis, prostatitis, impotence, and proctitis.

Corticosteroids are powerful pharmaceutical compounds that would prevent or diminish prostate gland enlargement/inflammation. Corticosteroids that would be useful within the context of the present invention include but are not limited to: corticosterone, cortisone, aldosterone, hydrocortisone acetate, methylprednisolone acetate or triamcinolone acetonide, dexamethasone sodium phosphate, betamethasone sodium phosphate and acetate, budesonide, hydrocortisone, methylprednisilone, prednisolone, prednisone, triamcinilone, alcometasone dipropionate, betamethasone valerate, desoximetasone, fluocinolone, flurandrenolide, fluticasone propionate, hydrocortisone butyrate, hydrocortisone valerate, mometasone furoate, amcinonide, betamethasone dipropionate, diflorasone diacetate, fluocinonide, halcinonide, clobetasol, diflorasone diacetate, halobetasol propionate, fluticasone, beclomethasone, flunisolide, halobetasol propionate, betamethasone valerate, clocortolone pivalate, fluocinolone acetonide, flurandrenolide, prednicarbate, triamcinolone acetonide, alclometasone dipropionate, and desonide.

A distinct class of anti-inflammatory pharmaceutical agents includes NSAID or Cox-2 inhibitors. Examples of NSAIDS include ketoprofen, mefenamic acid, nabumetone, diclofenac, diflunisal, indomethacin, sulindac, flurbiprofen, valdecoxib, celecoxib, rofecoxib, fenoprofen, etodolac, piroxicam, tolmetin, meloxicam, naproxen, oxaprozin, ketorolac, sulindac, phenylbutazone, ibuprofen, floctafenine, and meclofenamate.

Novel anti-inflammatory agents include but are not limited to minocylcline, colchicine, annexin 1, triptolide, interleukin-4 (IL-4), nuclear factor (NF)-κB inhibitors, licofelone, resvesterol, 5-furoyl-2,2,4-trimethyl-1,4-dihydro-1H-1,5-benzodiazepine, N-(substituted)-1-heteroaryl-oxindole-3-carboxamides (wherein the N-substituent is thienyl, furyl, phenyl or substituted phenyl), benzothiazine derivative, that is N-(2-pyridyl)-2-methyl-4-cinnamoyloxy-2H-1,2-benzothiazine-3-carboxamido 1,1-dioxide, 1-heteroaryl-3-acyl-2-oxindoles, substituted 2,3,4,9-tetrahydro-1H-carbazole-1-acetic acid derivatives, 1,5-diaryl pyrazole anti-inflammatory agents, human phosphlipase inhibitory protein (hPIP), substituted 2,3,4,9-tetrahydro-1H-carbazole-1-acetic acid derivatives, 4,5-diaryl-2-(substituted-thio)imidazols and their corresponding sulfoxides and sulfones.

The present invention may be implemented using any of the above-cited anti-inflammatory compounds. Presently-preferred embodiments employ corticosteroid anti-inflammatory agents. Triamcinolone acetonide (TAC), methylprednisolone acetate, and salts of betamethasone (such as the sodium phosphate and acetate derivatives) are particularly preferred. TAC would preferably be administered at a dose of about 10 mg to about 40 mg. Methylprednisolone is preferably administered at a dose of about 2 to about 10 mg. Salts of betamethasone is preferably administered at a dose of about 1 to about 3 mg.

Within the context of the present invention, the drug-eluting polymer would preferably be engineered to deliver the anti-inflammatory agent to the prostate gland over an extended period of time coinciding with the period of radiation delivery. As previously mentioned, literature reports that prostate brachytherapy induced enlargement has a half life of 30 days, meaning that for every 30 days that passes following the implant procedure, the enlargement will decrease 50% toward the eventual return to the normal size of the pre-implant gland. As stated above, this enlargement is caused primarily by surgical trauma but also has contribution from the radiation effect which may last for many months. When a ˜5 week course of external beam radiation therapy is combined with prostate brachytherapy, the radiation via I-125 seeds is delivered over an effective life of ˜10 months (or over ˜2 months with Pd-103 seeds).

Accordingly, the anti-inflammatory agents are preferably formulated such that they are released over a sustained period of time. The time over which the anti-inflammatory agent is released may vary from several days to several months in accord with the specific implementation of the invention that is employed. For example, in certain preferred embodiments, the anti-inflammatory agent is released into the prostate over the entire time that the radioactive seeds are exposing the prostate to radioactivity. In this manner, the anti-inflammatory drugs may reduce inflammation of the prostate that arises from exposure to radiation. In another example, benign prostatits, it may be preferable to have a sustained released over 6 months or longer.

The bioactive agent may be formulated in several manners. For example, the forms of injection, gel, foam, wafer, spray, solution, nanotechnology modalities, drug-eluting polymer, coated gel cap, or encapsulated solid dosage form are considered to be within the scope of the present invention. The bio-active agent may be either in an immediate-release or sustained-release formulation and employ a variety of vehicles or other compounds to change the rate of absorption, decay, or emission. Such properties may also be achieved by formulating the bio-active agent as a component of depot formulations, via infusion, or by mixing the drug with a polymer that may elute the bioactive agent.

The release characteristics of the polymer may be evaluated and chosen to optimize the concentration and temporal profile of the anti-inflammatory agent. A single immediate release, or relatively short-acting form (e.g., a single transperineal injection of soluble liquid form of the biologically active agent) injected directly into the prostate gland may not adequately or optimally prevent or reduce prostate gland enlargement. However, while such an approach of injecting a soluble drug directly into the prostate gland is not being used in current management strategies, such an approach of injecting a relatively shorter-acting form of a biologically active agent could potentially prove effective alone or in combination with the present invention (with drug delivery in a more durable form). In one present scheme, the bioactive compound (e.g., integrated into the polymeric matrix of “spacers”) would be released over 6 months to encompass the typical duration of prostate inflammation resulting from the prostate brachytherapy procedure. Of possibility, an initial transperineal injection of an anti-inflammatory drug (such as an injectable corticosteroid) could precede or accompany the placement of the bioactive compound in its more durable reservoir form (e.g., as “spacers”).

In presently-preferred embodiments, the bioactive agent is an anti-inflammatory agent and may be formulated in a sustained-release polymeric matrix. The sustained-release polymeric matrix may be comprised of any number of compounds that are well known in the art of pharmaceutical formulation. In certain preferred embodiments of the present invention, the controlled-release formulation is achieved via a polymeric matrix which includes a controlled-release material as set forth below. A dosage form including a controlled-release matrix provides in vitro elution rates of the anti-inflammatory agent within preferred ranges and that releases the anti-inflammatory agent in a pH-dependent or pH-independent manner. The dosage form may contain between 1% and 80% (by weight) of at least one hydrophilic or hydrophobic controlled-release material. The drug-eluting polymer may be bio-erodable or non-bio-erodable as desired for the particular implementation.

A non-limiting list of suitable controlled-release materials which may be included in a controlled-release matrix according to the invention include hydrophilic and/or hydrophobic materials, such as gums, cellulose ethers, acrylic resins, protein derived materials, waxes, shellac, and oils such as hydrogenated castor oil, hydrogenated vegetable oil. However, any pharmaceutically acceptable hydrophobic or hydrophilic controlled-release material which is capable of imparting controlled-release of the corticosteroid agent may be used in accordance with the present invention. Preferred controlled-release polymers include alkylcelluloses such as ethylcellulose, acrylic and methacrylic acid polymers and copolymers, and cellulose ethers, especially hydroxyalkylcelluloses (e.g., hydroxypropylmethylcellulose) and carboxyalkylcelluloses. Preferred acrylic and methacrylic acid polymers and copolymers include methyl methacrylate, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers, lactic acid polymers, glycolic acid polymers, polyglycolide, polylactide, poly-ε-caprolactone, poly-dioxanone, silicon, polyethelene, polypropylene, polyvinyl chloride, polyethylene terephthalate, polystyrene, and polycarbonate. Certain preferred embodiments utilize mixtures of any of the foregoing controlled-release materials in the matrices of the invention.

The formulation also may include a binder or other components to improve the properties of the material for the specific implementation. In addition to the above ingredients, a controlled-release matrix may also contain suitable quantities of other materials, e.g., diluents, lubricants, binders, granulating aids, colorants, and glidants that are conventional in the pharmaceutical art.

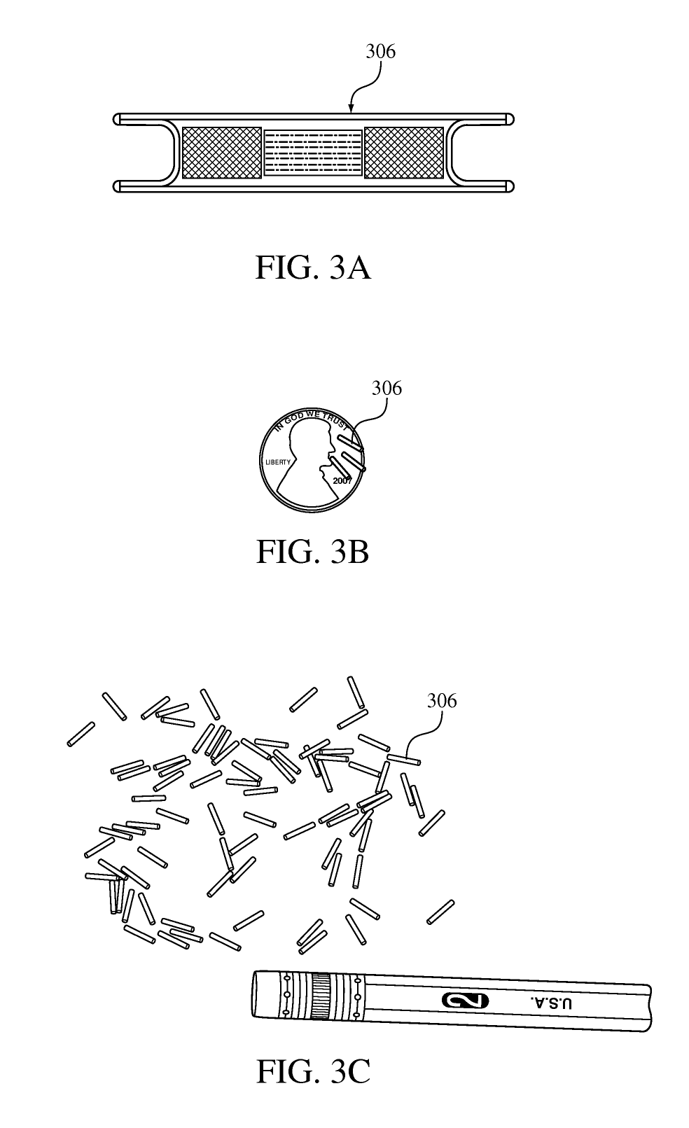

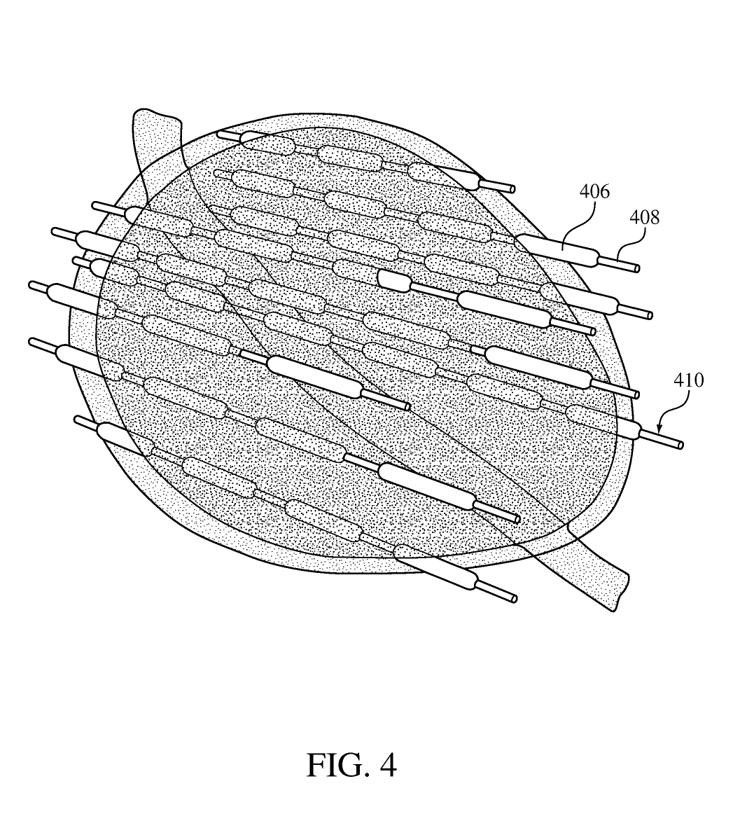

Of practical consideration, in order to seamlessly integrate into current prostate cancer management patterns and techniques, the present invention may preferably be employed as part of delivery methods currently and commonly used in prostate brachytherapy. In one embodiment of the present invention, a bioactive substance in short acting or long acting formulation could be used simply to coat the brachytherapy delivery needle. In one embodiment of the present invention, a bioactive substance-eluting polymer could be used simply to coat current radioactive seeds or non-radioactive “spacers”. In another presently preferred embodiment, the present invention could be embodied in the form of the inert biodegradable “spacers” currently and commonly utilized in pre-planned prostate brachytherapy procedures. In presently-preferred embodiments of the present invention, the biodegradable “spacers” are replaced with a polymer that slowly elutes the bioactive substance with local anti-inflammatory properties as shown in FIGS. 3 and 4. In another presently-preferred embodiment of the present invention, the bioactive substance may be incorporated into a biodegradable polymeric “sheath” that “welds” an entire array of radioactive seeds together in a fixed “string” with appropriate separation spacing between each radioactive seed (e.g., RAPID STRAND), as shown in FIG. 4. In yet another embodiment, the present invention encompasses a bio-absorbable “shell” whose breakdown would allow the subsequent local diffusion of the bioactive drug in the body of the prostate.

Importantly, if the present invention is embodied in the form of the inert biodegradable “spacers” currently and common utilized in pre-planned prostate brachytherapy procedures, it may used with either pre-planned, pre-loaded needles (alone or in combination with radioactive seeds) to implant the prostate gland or could be used with the Mick applicator in real-time intra-operative planned prostate seed implants.

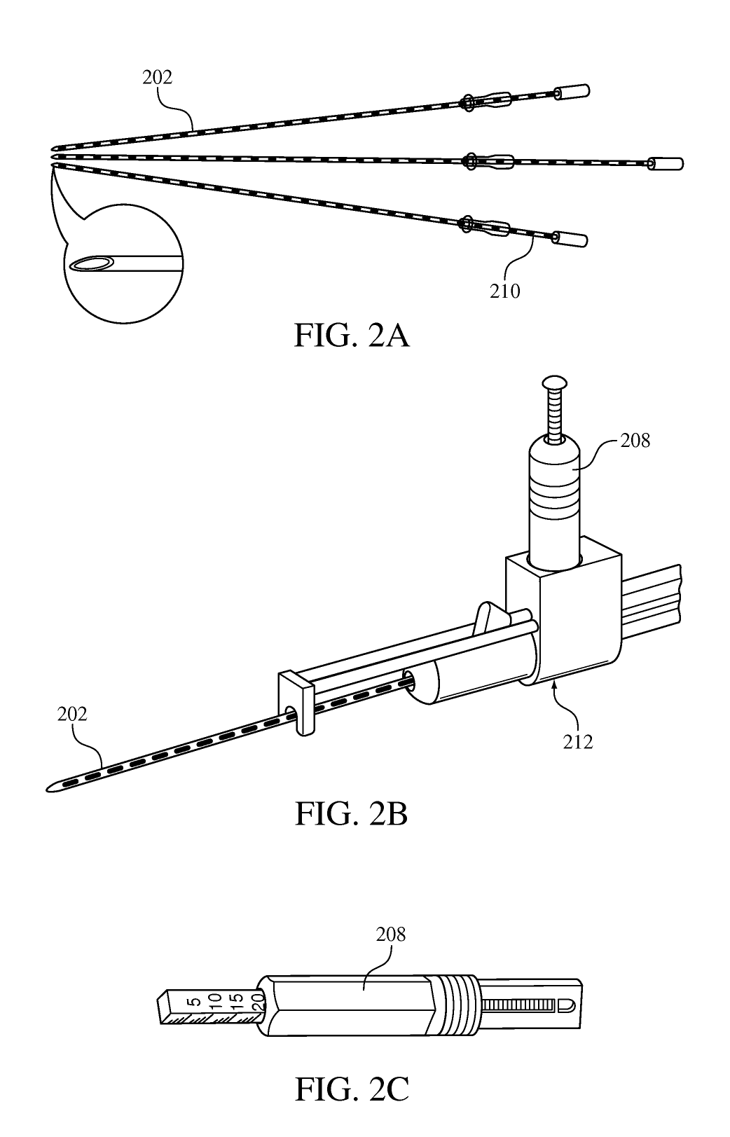

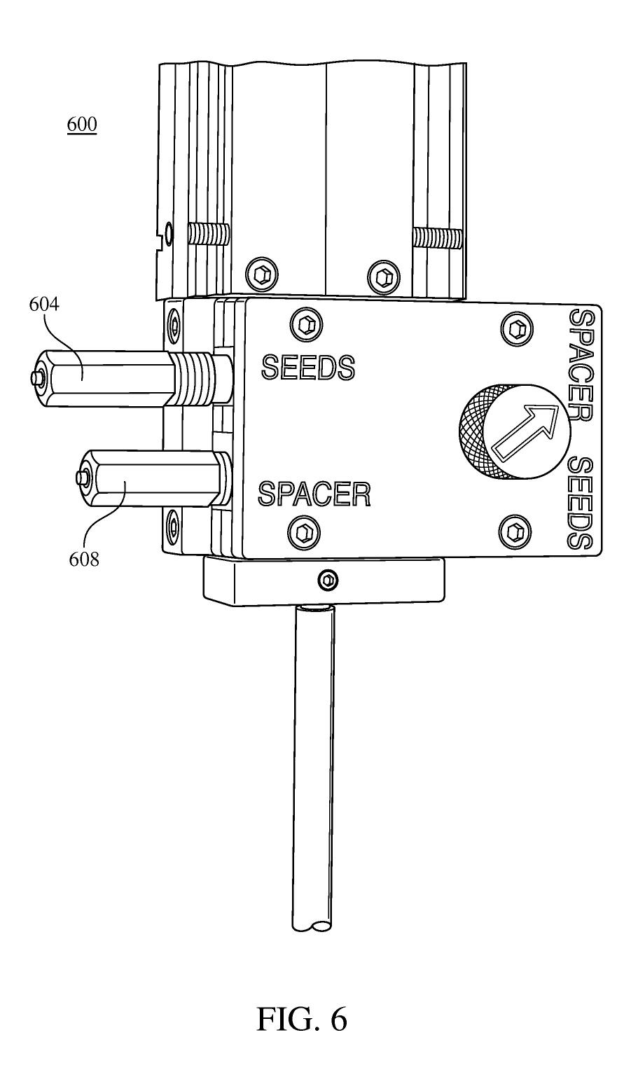

The Mick applicator is shown in FIG. 2. The present invention may be employed in the currently used seed cartridges for the Mick applicator, or alternatively with a novel design for an applicator or applicator cartridge that would be devised to utilize the present invention. The present design would preferably include dual cartridge chambers that could house both the seeds and “spacers” that preferably include a drug-eluting polymeric matrix, respectively, similar in design to the seed cartridge chamber currently in use in the existing Mick applicator system. An illustration of a possible dual chamber modification for the existing Mick applicator is shown in FIG. 6 where the novel applicator gun has one chamber that houses the radioactive seeds and a second chamber that holds the drug-eluting “spacers”. Alternatively, the second storage device rather than being a chamber may be a syringe that holds a depot solution of drug or corticosteroid, in which case a selector switch would be utilized in order to open one chamber and subsequently close the second to allow only one drug composition to be administered at any single time. Additionally, there could be a third setting on the switch that would open both storage devices and allow one of each type of seed or liquid suspension to be emitted simultaneously.

However, intraoperative brachytherapy techniques need not employ the Mick applicator to deposit seeds or a drug-eluting spacer one by one. As is known in the art, it is possible by using ISOSTRAND or related technology to “manufacture” strands of seeds and spacers in 30 seconds in the intra-operative setting.

The present invention may also be used with high dose rate (HDR) brachytherapy. The catheters implanted in the operating room as part of HDR brachytherapy may be coated or impregnated with a bioactive substance (e.g., anti-inflammatory agent) as described above. Alternatively, bioactive substance could be injected or deposited in some other fashion prior to or preferably at the time of implantation of the HDR catheters. Also, any treatment planning system that would incorporate into consideration the bioactivity of the substance in the prostate gland as described above for LDR brachytherapy techniques would apply with appropriate modification for HDR brachytherapy techniques.

The present invention may also be employed using as part of image-guided techniques presently employed in the art. Depending on the release characteristics of the embodiment of the bioactive drug defined by the present invention, image-guided techniques would likely be employed to properly localize and place such bioactive drug embodiment appropriately within the prostate gland in order to volumetrically “saturate” the gland for optimal clinical effect. Of convenience, the bioactive drug embodiment may be designed to be radiographically (e.g., on ultrasound, fluoroscopy, CT scan, MRI) visible in situ but visibly distinct from radioactive seeds (or, if needed, from other bioactive drug embodiments that are placed).

The present invention also encompasses the use of treatment planning systems for dose optimization relates to anti-inflammatory eluting seeds alone or in conjunction with radiation seeds. Along the lines of current treatment planning systems for radioactive seeds, the creation of a novel treatment planning system or modification of current radiation seed treatment planning systems would be employed to define the appropriate spatial distribution of the drug-eluting polymers placement within the prostate gland. Such systems may be used to account for the diffusion properties of corticosteroid within the prostate gland which may vastly differ from the radiation dose emission properties of radioactive seeds. Further, the placement of drug-eluting polymers within the prostate gland may be preferential to the peri-urethral region which may prove most efficacious in reducing urinary morbidities such as obstructive urinary problems or urethritis. The present invention further relates to the use of computer software for pre-planning, intraoperative planning and post-planning assessment of the dosimetry of drug-eluting seeds either solely or in conjunction with radiation brachytherapy.

In alternative implementations of the present invention, the bioactive agent may be deposited directly either by injection, or gel coated or capsule format rather than through a drug-eluting polymeric matrix. The gel or capsule may dissolve thus releasing the bioactive compound. The seeds may contain medicine in concentrated or diluted formula or in a vehicle. Also, the bioactive agent could be in depot long-acting, short-acting or combination formulation. The spatial release profile of the bioactive agent release may be determined and entered into the computer software, much as the spatial exposure of radiation seed sources is calculated for the treatment of prostate cancer. Based on this dosing the computer can determine the dose volume histograms and doses to all tissues (bladder, rectum, etc.) and the optimal arrangement of any radiation or bioactive substance sources. The use of the treatment planning system for bioactive substances elution, gel, depot, or other forms would be used to plan an approach to maximally decrease morbidity. This would especially be the case in the peri-urethral area where inflammation often leads to urinary complications and catheterization as a side effect—or near the neurovascular bundles to prevent impotence.

In summary, it is proposed that local administration of an anti-inflammatory agent (such as a corticosteroid) into the prostate gland may prove effective in preventing or reducing radiation and surgical trauma-related enlargement of the prostate gland following external beam radiation therapy and/or prostate brachytherapy. A reduction of the inflammatory enlargement of the prostate gland would allow for the preservation of initially planned dose delivery parameters, which in turn could result in improved disease control and decreased patient morbidity to allow for improved patient comfort as well as improved disease local control and survival. Additionally, as discussed above, anti-inflammatory agents could independently reduce radiation-induced local morbidites such as radiation urethritis, bladder neck spasticity, and prostatitis. Relating to the adverse effects of radiation on surrounding peri-prostatic areas, local anti-inflammatory agents delivered to the prostate gland could potentially reduce the incidence of impotence (related to damage to peri-prostatic neurovascular bundles) and radiation proctitis (for which corticosteroid suppositories have been shown to be effective and are currently in clinical use for proctitis).

The present invention provides numerous benefits to the art. A significant clinical benefit gained from the prevention/reduction of prostate gland enlargement would be a decrease in obstructive urinary morbidity (e.g., obstructive urinary flow symptoms) for the patient commonly resulting otherwise from the narrowing/compression of the urethra through which urine flows.

Another significant clinical benefit gained from present invention would be the prevention or reduction of radiation morbidity (e.g., radiation proctitis or tenesmus, radiation urethritis (causing dysuria and/or hematuria), bladder neck spasticity (causing urgency, incontinence, etc.), prostatitis, or impotence).

Another significant benefit of the present invention may be improved (adequate and homogenous) dose delivery to a prostate gland more consistent in size with pre-planed or intra-operative volume specifications for radioactive seed activity, quantity, and seed localization.

In addition to radiation or surgical (e.g., brachytherapy) related trauma to the prostate gland, systemic androgen blockade therapy may be responsible for prostatic scarring and fibrosis. Such scarring/fibrosis could manifest as obstructive urinary symptoms. Bioactive substances (such as corticosteroids) deposited locally within the prostate gland may provide the additional benefit of preventing scarring/fibrosis related to androgen blockade therapy and/or brachytherapy/external beam radiation—or diminishing/reducing existing scarring/fibrosis within the prostate gland.

Prior to patients undergoing external beam radiation therapy or at the time of brachytherapy, the surgical injection of amifostine or corticosteroid or any other cytoprotective or anti-inflammatory agent into the neurovascular bundles of the prostate prior to treatment may protect against nerve damage and decrease morbidity. This will lead to decreased impotence. It can elute over time to protect during the whole course of treatment for example during the 6-8 week course of external beam or during brachytherapy. These patients often are potent and lose potency over time so the invention would consider possible mechanisms and ways to counteract these. Nerve stimulation studies even during prostatectomy show that the nerves often work but impotence is still present. There are 3 mechanisms by which impotence is caused: 1) radiation dose (especially to penile bulb) 2) trauma 3) vascular injury. Note that this technique would not likely cause any sequelae or scarring as currently the neurovascular bundles are sometimes injected for local anesthesia during transrectal ultrasound biopsy. It has been shown that this technique does not interfere with or have a higher incidence of adverse effects when then these patients subsequently undergo nerve sparing radical retropubic prostatectomy (Prostate Cancer Prostatic Dis. 2003, 6(1):53-5).

As is well known in the art, suitable brachytherapy source seeds may be composed of various radioactive isotopes including but not limited to lower energy iodine-125 seeds, consisting of a welded titanium capsule containing iodine-125 isotope, or higher energy palladium-103 seeds. Additionally, other seed isotopes include gold-198, cesium-131, iridium-192, phosphorus-32, radium-226, and radon-222, cobalt-60, and yttrium-90. Other types of seeds can be used such as those described in U.S. Pat. No. 6,248,057 to Mavity et al., which is incorporated herein by reference. Furthermore, any radioactive isotopes including but not limited to the ones above can be incorporated into mixtures with various bioactive or inert polymers or substrates and can be use to form secondary compounds which degrade eluting the above isotopes over short or long period of time. These polymers can degrade and disappear over time or have remnants that remain active within the body over time.

Importantly, the present invention would additionally allow a platform for the coupling of several different classes of drugs within the context of presently-employed surgical (brachytherapy) delivery techniques. The present invention could be devised to elute a combination of several compounds together such as an anti-inflammatory agent and an alpha blocker, or even a combination of an anti-inflammatory agent and a chemotherapeutic agent. Additionally, the present invention could utilize two similar classes of compounds in conjunction with another class of agent (e.g., two discrete anti-inflammatory drugs in combination with several molecular targeting agents).

As noted, the present invention also encompasses formulations where an anti-inflammatory corticosteroid is co-administered with other agents. Examples of such agents are listed hereafter.

Anti-inflammatory agents may be co-administered with antimicrobial agents such as neomycin, polymyxin, fusidic acid, clotrimazole, and nystatin to reduce the likelihood of infection. Anti-infective (antibiotic/antifungal) agents including but not limited to cephalosporins, tetracyclines, macrolides, quinolones, linezolid, imipenem, miropenem, fluconazole, nystatin, veroconazole, itraconazole, and terbinafine.

Furthermore, drugs used to treat the resulting symptoms such as urinary flow obstruction or inadequacy, intermittency, dysuria, hematuria, or rectal irritation and bleeding could potentially be used in local application within the context of the present invention in combination with anti-inflammatory agents described in the present invention. Such “symptomatic” drugs include uroselective alpha-blockers (e.g., tamsulosin, alfuzosin), anti-cholinergics (e.g., oxybutynin, tolterodine, etc.) or anti-spasmotic agents (e.g., solfenacin). Other potential classes of agents that might prove effective against radiation or surgically induced inflammation would include agents that prevent or diminish capillary leakage or enhance any of the various forms of tissue repair (fibrinogen deposition, collagen synthesis, etc.) associated with the natural enlargement of the healing tissue.

Additionally, various bioactive compounds may have therapeutic efficacy either directly or indirectly on prostate cancer cells. Specifically these would likely be most effective: Chemotherapeutic agents—Docetaxol, Mitoxantrone; the preferred corticosteroids are indicated below with potential dosages; preferred cytoprotective agents—amifostine; of the hormone agents the anti-androgens would be most preferred like Flutamide, Bicluatmide then followed by the alpha reductase inhibitors like Finasteride, Dutasteride; the targeted therapies (molecular agents like small molecule or monoclonal antibody) also could be quite effective especially a monocolonal antibody against PSMA (prostate specific membrane antigen), the targeted therapies plus anti-androgen agents combined could be especially effective as explained in our embodiment paragraphs;

gene therapy agents targeted to androgen receptor down regulation, immunostimulant agents

Additionally, any class of agents proving cytoprotective for benign or normal prostate cells while allowing for enhanced tumor killing of malignant cells would be applicable to the present invention.

Suitable agents for the innovations and embodiments include chemotherapy, hormone, anesthetics, gene therapy, vaccines, immunotherapy, monoclonal antibody agents, small molecule agents clotting agents, anticoagulation agents, antibiotic agents, anti-scarring, anti-fibrotic, anti-inflammatory and cytoprotective drugs and drug classes. Some suitable agents that can be used include but are not limited to the following:

Antibiotic agents such as bleomycin, dactinomycin, doxorubicin, and mytomycin C.

Alkylating agents include but are not limited to busulfan, carboplatin, carmustine, chorambucil, cyclophosphamide, ifosfamide, lomustine, mechlorethamine, melphalan, oxaliplatin, procarbazine, and temozolomide.

Atypical alkylating agents include but are not limited to cisplatin, satraplatin, and carboplatin.

Antimetabolites agents include but are not limited 5-fluorouracil, capecitibine, cytarabine, fludarabine, gemcitibine, hydroxyurea, and methotrexate.

Enzyme agents such as L-asparaginase.

Endothelin receptor antagonist agents include but are not limited to xinlay.

Monoclonal antibody agents include but are not limited to trastuzamab, alemtuzumab, bevacizumab, cetuximab, gemtuzumab ozogamicin, rituximab, panitumumab, and pertuzumab.

Radioimmunotherapy agents ibritumomab tiuxetan and tositumomab.

Small molecule agents (such as tyrosine kinase inhibitors) include but are not limited imatinib mesylate, erlotinib gefitinib, lapatinib, disatinib, nilotinib, and sunitinib.

mTor inhibitor agents useful in the present invention include but are not limited to rapamycin, sirolimus, zatarolimus

Immunotherapy agents include but are not limited provenge, GVAX, and imiquimod.

Hormonal agents may also be used within the context of the present invention in the standard care of prostate cancer or prostate inflammation. Listings of various members of that group are listed as follows. LHRH (leutinizing hormone-release hormone) agents include but are not limited to goserelin acetate, leuprolide acetate, and leuprorelin acetate. Alpha reductase inhibitor agents include but are not limited to finasteride, dutasteride. LH receptor antagonist agents include but are not limited to plenaxis. Nonsteroidal antiandrogen agents include but are not limited to flutamide, bicalutamide, tamoxifen, nilandron, and eulexin.

Taxane agents include but are not limited to docetaxel and paclitaxel.

Topoisomerase inhibitor agents include but are not limited to etoposide, etoposide phosphate, and irinotecan.

Vinca alkaloid agents include but are not limited to vinblastine, vincaleukoblastine, vincristine, leurocristine, VCR, vinorelbine, and 5′-noranhydrovinblastine

Alpha blocker agents include but are not limited to doxazocin, terazosin, tamsulosin, and alfuzosin. These agents are presently-employed in the treatment of benign prostatic hypertrophy for obstructive symptoms and may be included with the local drug delivery formulations disclosed herein. These formulations would be particularly useful in the periurethral prostate area to prevent/relieve post seed implant obstructive symptoms.

Anticholingergic agents include but are not limited to tolterodine, flavoxate, and oxybutinin. These compounds may be employed within the context of the present invention to treat symptoms of bladder spasm especially in the part of the prostate implanted near the bladder neck.

A single injection of botulinum-toxin (Botox) via ultrasound guidance into the prostate gland has been shown in a recent study published in the British Journal of Urology to be effective for up to 1 year in managing benign prostatic hypertrophy (BPH) causing a 30% decrease in urinary symptoms. This can be used as a treatment for BPH in place of daily or twice a day alpha blocker pills. In one presently-preferred embodiment of the present invention, botulinum toxin can be deposited in a depot form or polymeric matrix to elute over time either at the time of prostate biopsy or at the time of seed implant (or at the time of the other aforementioned procedures). It could be deposited independently or in combination with other substances such as coritcosteroids. The mechanism of action is thought to be through a blockage of nerves which contribute to prostate cell proliferation.

Cytoprotective agents include but are not limited to amifostine.

Hormonal agents may also be used within the context of embodiments of the present invention in the treatment of breast cancer. Listings of various members of hormonal agents are listed as follows. Nonsteroidal aromatase inhibitor agents include but are not limited to anastrazole, letrozole, vorozole, and cytadren.

Steroidal aromatase inhibitor agents include but are not limited to exemestane.

SERM (selective estrogen-receptor modulators) agents include but are not limited to tamoxifen, evista, fareston, and arzoxifene.

Mixed estrogen agonist/antagonist agents include but are not limited to Clomiphene.

Hormonal therapy agents include but are not limited to faslodex and megace.

The present invention may also be employed within the context of gene therapy applications. Gene therapy techniques are well known in the art and include but are not limited to somatic (in vivo or ex vivo) or germline approaches these could be accomplished using vectors and methods including but not limited viral vectors (viruses, retroviruses, adenoviruses, adeno-associated viruses, envelope protein pseudotyping of viral vectors) and non-viral methods (naked DNA, oligodeoxynucleotides, lipoplexes and polyplexes) and hybrid methods.

Cytokine agents include but are not limited to those composed of peptides, proteins, and glycoproteins and whose examples include but are not limited to lymphokines, interleukins, and chemokines.

Agents which target cytokine receptors include but are not limited to immunogloubulins, hematopeoetic growth factor, interferon, tumor necrosis factor (TNF), and seven transmembrane receptors.

To prove efficacious, the aforementioned substances maybe used alone or in combination with additional bioactive substances. These include monoclonal antibodies or small molecules against HER2, EGFR, Prostate Specific Membrane Antigen (PSMA), VEGF or any other molecule or receptor, as well as anti-HER2 (trastuzumab) and anti-EGFR (cetuximab and panitumumab) monoclonal antibodies and anti-EGFR kinase inhibitors (Erlotinib and Gefitinib), Lapatinib, a HER2 kinase inhibitor, HER2 dimerization inhibitor (Pertuzumab).

Mixtures of any or multiple chemotherapy, hormone, anesthetic, gene therapy, vaccine, immunotherapy, monoclonal antibody agents, small molecule agents clotting agents, anticoagulation agents, anti-infective and antibiotic agents, anti-inflammatory, and cytoprotective drugs including but not limited to those mentioned above can be made with different vehicles, compounds, powders and solutions to produce depot and sustained-release formulations. Furthermore, mixtures with various bioactive or inert polymers or substrates can be use to form secondary compounds which are biodegradeable or nonbiodegradeable or soluble or insoluble which elute the above substances over time. These polymers can degrade and disappear over time or have remnants that stay within the body over time and have activity of their own. Alternatively the above bioactive or inert substances can be formulated with any particles or nanoparticles including but not limited to powder, nanopowder, nanosphere, microsphere, micropowder, microbead, liposome, and beads. Varying dosages may be used to maximize efficacy.

The bioactive drug could be formulated such that it is either activated by or released/eluted from the polymer when stimulated by some form of energy. Examples of forms of energy include but are not limited to heat, radiation (external beam or brachytherapy), electrical (electrocautery, transdermal transducer, etc.), or ultrasound. One preferred embodiment would be a polymer that elutes a drug that is activated when exposed to radiation.

The bioactive substance could be injected in a specific formulation and spatial arrangement appropriate for each specific tumor type and location such that it could be used for concurrent chemotherapeutic and radiation treatments similarly in concept to the management of head and neck tumors, where systemic chemotherapy is given as a radiosensitizer, only with this approach, the chemotherapy would be deposited locally and only be active during the time of radiation delivery or shortly thereafter—thereby maximizing efficacy and minimizing side effects. Another presently preferred embodiment is the deposition of bioactive substance prior to treatment with high frequency ultrasound prostate ablation. The ultrasound would activate the bioactive substance which then would lead to them working synergistically to destroy tumor cells.

Additional embodiments of the present invention could result in improved outcomes in other diagnostic or therapeutic approaches to the prostate. Some of the areas where a significant benefit could be derived include the treatment of benign prostatic hypertrophy (BPH), at the time of prostate biopsy, during radical retropubic prostatectomy for prostate cancer, and relating to other surgical approaches (e.g., robotic prostatectomy or laparoscopic prostatectomy) relating to the prostate gland.

In an embodiment of the present invention relating to BPH, biological substance eluting “seeds” or related formulations could be applied either via the current radiographically (e.g., ultrasound- or MRI-) guided brachytherapy techniques used in malignant prostate disease management or via currently used transurethral approaches for BPH. These transurethral approaches for BPH management include microwave, laser, or surgical therapies (e.g., transurethral resection of the prostate (TURP)). The urologist performing these transurethral procedures would have the ability to deposit biological active drug-eluting “seeds” or various depot formulations into the periurethral substrate of the gland. These “seeds” may be inserted using modified cystoscopy equipment or a novel version of a resectoscope currently used for urological procedures. A potentially effective drug formulation might include anti-inflammatory agents (e.g., corticosteroids), hormone antagonists (e.g., bicalutamide, finasteride, dutasteride, etc.), alpha-blockers (e.g., tamsulosin, alfuzosin, etc.), anti-cholinergics (e.g., oxybutynin, tolterodine, etc.), and procoagulant agents. Acutely, these substances would minimize the inflammation and bleeding (which are the major contributors to morbidity for these patients), and chronically, might prove active biologically effective in reducing existing hypertrophy (or preventing re-hypertrophy). These biological agents would facilitate rapid recovery, decrease post-operative pain and discomfort, and promote early removal of the Foley catheter.

In another preferred embodiment of the present invention, drug-eluting polymers may be used at the time of either open prostate surgery (e.g., radical retropubic prostatectomy) or laparoscopic or robotic prostatectomy. These polymers may be formulated into beads, foam, wafers, gel, spray, thin film, or other formulations and would be placed in the prostatic and periprostatic tissues at the time of surgery. These polymers would elute biological substance(s) to promote post-operative healing and decrease the significant morbidity associated with radical prostatectomy. The use of anti-inflammatory agents or other neuroprotective biological substances could potentially improve the regeneration of nerves in the prostatic nerve bundle thereby reducing the incidence of impotence.

Utilizing the abovementioned transurethral drug formulation deposition techniques described with BPH, another embodiment of the present invention may be used at the time of diagnostic prostate biopsy. Prostate biopsy can be transperineal or transrectal. Various biopsy techniques include standard biopsy as commonly in clinical use, or more extensive biopsy techniques such as mapping biopsy (which is performed under ultrasound guidance at 5 mm intervals using a mapping grid) or saturation biopsy (much more extensive sampling of the gland). Commonly performed via transrectal ultrasound-guided approach under local anesthesia, the biopsy procedure is often quite painful both during and after the procedure completion (as biopsy “bites” are removed from the prostate gland). Patients often experience untoward hematuria, dysuria, and obstructive urinary symptoms (sometimes requiring extended Foley catheterization) in the post-biopsy period. The obstructive symptoms result from biopsy related trauma and inflammation of the prostate gland. A biologically effective substance eluting polymer or depot preparation to reduce morbidity could be left behind in the substrate of the prostate gland at the time of biopsy. The substances in the eluting polymer or depot preparation could be a combination of corticosteroids, alpha-blockers, hormones, anesthetic/analgesic medications, or other bioactive substances as noted hereinabove.

In a related implementation, biopsy localization markers or fiducial markers could be deposited into the prostate gland (or in any other site in the body) at the time of prostate biopsy or any other procedure to optimize treatment planning and patient localization for radiation therapy. Alternatively, these markers could be placed at a separate time in areas where there is relative immobility to optimize their use as fiducial markers for radiation therapy localization. Various biopsy location markers could be differentiated from one another to correlate certain biopsy specimens with their location in the target organ (e.g., prostate). These markers would be differentiable via the use of noble gas rings that would be localized and identified via imaging. Additionally a saturation technique for placing biopsy markers could be used to determine the full extent of tumor area positivity as seen on imaging. This technique would be particularly useful in areas where the true extent of tumor is difficult to differentiate from necrosis. Fiducial markers would be essential in an image guided radiation therapy (IGRT) system similar to the one used in the prostate. These markers could also be used for local bioactive substance treatment delivery to localized areas of tumor that were positive on biopsy.

In another preferred embodiment, the deposition of bioactive substances may be performed in conjunction with cryotherapy. Bioactive substance deposition could be employed either prior to, after, or preferably during the procedure. In one preferred embodiment the bioactive substance would be corticosteroids to prevent inflammation and morbidities related to this procedure. Cryotherapy utilizes sub-zero temperatures to achieve tumor destruction. Cryotherapy is performed by inserting cryoprobes or cryoneedles into target tumor tissue typically under image guidance (e.g., transrectal ultrasound). That procedure is subsequently followed by the circulation of a freezing gas such as liquid nitrogen into the probes or needles. The freezing gas is used to create ice balls around the probes or needles leading to tumor destruction. In newer generation cyrotherapy techniques helium or argon gases are often used along with a warming catheter. In one preferred embodiment to be used with cryotherapy the probes, needles, catheters or any other invasive tool which is inserted into the body during the procedure could be coated with a bioactive substance such as corticosteroids. Alternatively, bioactive substance could be injected or deposited in some other fashion prior to or preferably at the time of cryotherapy. In another presently preferred embodiment the cryoprobes or needles which are currently used to circulate freezing gas into the target tissue could be modified such that they could also be utilized to inject or deposit bioactive substance into said target tissue. One presently preferred modification of this apparatus would be to connect the cryoprobe end to a reservoir that houses bioactive substance in one of the following preferred forms (liquid, gel, foam, eluting polymer, etc.).

Other minimally-invasive therapeutic techniques with which bioactive substance deposition could be used throughout the body include radiofrequency ablation (RFA), percutaneous stereotactic excision or biopsy, photodynamic therapy, or laser ablation. Bioactive substance deposition could be employed either prior to, after, or preferably during the above mentioned procedures. In RFA, under image guidance (ultrasound, MRI, etc.) the RFA probe (e.g., 15-gauge) is inserted into the center of the lesion and a star-like array of electrodes is deployed from the tip of the probe. Subsequently, an alternating high frequency electric current (400-500 kHz) is administered. This leads to thermal tumor destruction as tumor cells are more susceptible to heat than are normal cells. In one presently preferred embodiment of the present invention related to RFA the probe, electrodes as well as any other invasive tool employed in the procedure could be coated with bioactive substance (e.g., corticosteroid). In another presently preferred embodiment the RFA probe could be modified to deliver bioactive substance into the target tissue. Radiofrequency ablation has been used successfully for the treatment of primary and metastatic tumors of various sites including liver, lung, bones, central nervous system, pancreas, kidneys, prostate, and breast. In another embodiment of the present invention, during laser ablation procedures, photodynamic therapy, or stereotactic biopsy/excision procedures bioactive substances (e.g., corticosteroids) can coat the various needles, probes, catheters or other invasive devices utilized during these procedures. Alternatively, bioactive substance could be injected or deposited in some other fashion prior to or preferably at the time of any of the hereinabove mentioned minimally invasive procedures. Furthermore, any devices (e.g., clips, sutures) left in the body could be coated with bioactive substance.

In another preferred embodiment bioactive substances could be deposited during high frequency focused ultrasound ablation (FUA) of tumors within the body (e.g., prostate). Bioactive substance deposition could be employed either prior to, after, or preferably during the procedures. FUA uses ultrasound acoustic energy and converts it into heat to kill tumor cells. Since this procedure can be completely noninvasive, bioactive substance deposition could be performed either during the procedure or at a separate time. In a presently-preferred embodiment bioactive substances could coat or be deposited with the temperature monitoring probes which are often inserted into the target tissue to guide the thermal ablation involved in this procedure. Alternatively, bioactive substance could be injected or deposited in some other fashion prior to or preferably at the time of the FUA.

In an application related to breast cancer bioactive substances can be deposited within the breast and surrounding tissue in conjunction with either diagnostic or therapeutic techniques. In relation to diagnostic techniques in breast cancer one presently preferred embodiment would be to coat the needles or invasive tools involved in fine needle aspiration (FNA), large-core needle biopsy, vacuum-assisted needle biopsy or needle localization biopsy. In relation to therapeutic surgical techniques in breast cancer bioactive substance can be deposited in the form of a gel, wafer, foam, mesh or hardening substance into the resection site (lumpectomy, mastectomy, etc.) at the time of surgery. In one presently-preferred embodiment these bioactive substances (chemo, hormonal agents, steroids, radioactive isotope, etc.) deposited at the time of surgery can be eluted over a period of weeks to months. In furtherance of this embodiment the elution period of certain bioactive substances (e.g. steroids) can coincide with the duration of adjuvant radiation therapy (external beam, mammosite, etc.) given to the patient. In another preferred embodiment in breast cancer bioactive substances (hormonal agents, steroids, etc.) can be used during breast brachytherapy. In this presently preferred embodiment of the invention bioactive substances can either coat the catheters or the mammosite balloon commonly used in breast HDR brachytherapy.

As noted above, the present invention may also be useful in any application where surgical intervention or radiation administration results in the inflammation of a target tissue, or where further therapeutic gain would result from the deposition of a bioactive substance into the resection or surgical site. A radioactive isotope/chemotherapeutic-agent eluting polymer could be placed in the resection cavity. Also, bioactive substance could be deposited at the time of biopsy or any other invasive procedure. In one preferred embodiment of the present invention the biopsy needles, probes, or any invasive tools could be coated or used to inject bioactive substance at the time of biopsy. Similar to the implementations in prostate, this embodiment of the present invention would allow radioactivity, chemotherapeutic agents, and/or anti-inflammatory agents to elute over time into the target tissue. Furthermore, similar to the embodiment of the current invention relating to the prostate, radiation therapy (external beam, brachytherapy, etc.) in any form could be used in conjunction with the deposition of bioactive substances throughout the body. Additionally, any treatment planning system which would utilize the dosing and spatial arrangement of any deposited bioactive substances to formulate a dosimetric treatment plan for the delivery of radiation would also be included within the scope of the current invention.

Relating to neurosurgical procedures within the brain, in one presently preferred embodiment the bioactive substances deposited would be a combination of chemotherapy (e.g Temezolomide) and anti-inflammatory (e.g. cortocosteroids) agents. In a particularly preferred embodiment of the present invention the bioactive substances would be formulated to elute over several months to encompass the period of the patient's course of adjuvant or definitive radiation therapy. In another preferred embodiment of the invention the aforementioned bioactive substances would be placed either via biopsy or open surgical procedure such that they are in continuation with the circulating cerebrospinal fluid (CSF). This embodiment of the present invention would allow for local therapeutic efficacy throughout regions of the brain and spine in continuation with the CSF, while minimizing systemic effects. Similarly, the present embodiment encompasses the use of an anti-inflammatory agent-eluting substance or depot gel/foam/wafer or any substance in neurosurgical procedures of the brain or spine in biopsy, resection, or other invasive or non-invasive procedure. Inflammation is a significant problem in these procedures—particularly in such confined spaces such as the skull or spinal cord—as it may lead to the compression of critical structures with resultant neurological symptoms. Local deposition of such substances may prove more effective than current clinical measures such as the use of systemic steroids (oral or IV) such as Decadron to reduce such inflammation and edema. The local deposition of such anti-inflammatory substances may be performed through any procedures described herein. Potential formulations of anti-inflammatory agents for local deposition would include those mentioned in U.S. patent application publication no. 20050059615.

Orthopedic procedures (i.e., joint replacement with prosthesis) are associated with untoward side effects—namely heterotopic osteopathy (i.e., abnormal/hypertrophic bone formation) adjacent to the prosthetic device. Such reactive bone formation can lead to significant joint pain on movement. Relating to orthopedic procedures, the local placement of a bioactive substance (e.g., corticosteroid) to prevent heterotrophic bone growth may improve on current clinical strategies which involve systemically-administered high dose NSAIDS or involve a single fraction of external beam radiation therapy prior to the surgical procedure. The bioactive substance in this embodiment of the present invention could be a radioactive isotope, an anti-inflammatory agent such as a corticosteroid, another efficacious agent, or combination of agents. The bioactive substance could be applied directly onto the orthopedic hardware as a gel, sticky spray, or surface coating. A specific implementation where this technology may be highly applicable would be hip replacement surgeries. Local application of a bioactive substance would likely prove more effective than systemic administration of such drug. Additionally, with the current use external beam radiation therapy to prevent heterotopic osteopathy in this site, a major concern is the damaging effect of radiation on the skin (i.e., patient immobility post operatively often leads to chronic indolent pressure ulcers especially in radiation exposed areas). An internal radioactive or bioactive drug-eluting substance could avoid this potential complication.

In an additional related application bioactive substances (e.g., corticosteroids) can be used to coat any device or object placed within the body (temporary or permanent). In one embodiment of the present invention the device itself could be composed of a polymer or any substance such that it would decay and be absorbed by the body over time and concurrently release a bioactive substance over said time. Examples of devices or objects which could be coated or infused with bioactive substances include but are not limited to stents (cardiovascular, urethral, ureteral, etc.), orthopedic implants, heart valves, pacemakers, neurosurgical implant, ophthalmic implants, sutures, staples, clips, nails, and screws.

Kyphoplasty or vertebroplasty relates to the orthopedic/neurosurgical repair or reconstruction of vertebral bodies that often lose structural integrity secondary to compression fracture as the result of osteoporosis or metastatic disease. Kyphoplasty is typically performed by insertion of trocar and subsequent inflation of a balloon. The balloon decompresses the vertebral body and opens a potential space which is then filled with cement. Vertebroplasty similarly accomplishes the same thing without the use of a balloon, but with high pressure cement which is injected into the cavity to decompress and stabilize the vertebrae fracture. Both procedures serve to mechanically stabilize the compression fractures and relieve pain from the fracture. However, the patient subsequently has to undergo external beam radiation for the bony metastases to relieve the pain caused by tumor. If there was the incorporation of a bioactive substance within the cement which would serve to locally kill the tumor cells then the patient would not have to undergo the two weeks of subsequent palliative radiation therapy. Such patients often have short life expectancies secondary to extensive metastatic disease often on the order of months, so a procedure which would palliate the pain from the tumor as well as the fracture all in one procedure would be optimal for these patients. Incorporation of a bioactive substance (e.g., chemo-eluting or radiation-eluting substance such as Sm-153 or Sr-89) within the “bone cement” used for kyphoplasty will result in improved tumor cell kill and lasting efficacy of treatment. Additional bioactive substances which could be utilized in this preferred embodiment of the present invention would be any bioactive substance which would physiologically encourage bone growth (e.g., bisphosphonate).

Another related embodiment of local deposition/elution of bioactive substance (e.g., corticosteroids) over time would include ophthalmologic interventions such as intraocular surgery or any other invasive procedure performed on the eye. Examples of such procedures would include cataract extraction, glaucoma filtering surgeries, vitrectomies, keratoplasty, traumatic open globe injuries, corneal laceration repair. One preferred embodiment would encompass the deposition of a bioactive compound during the procedure such as a corticosteroid or anti-adhesion/anti-scarring agent to elute over time to reduce inflammation and prevent synechiae. This bioactive compound could be in the form of a gel, mesh, wafer, foam, or hardening substance left in place at the time of surgery. Another embodiment would be the coating of any device that would left in place within the globe—such as an IOL (intraocular lens). Currently, to prevent untoward reaction/side effects after such procedures, these patients are managed with steroid drops (4 times a day tapered over a month), but such administration proves highly inconvenient for many patients and leads to an increased risk of complications (including a decrease in visual acuity) for noncompliant patients.

Those of skill in the art will recognize that numerous modifications of the above-described methods and apparatuses can be performed without departing from the present invention. For example, one of skill in the art will recognize that various combinations of bioactive substances and various drug-eluting polymers may be employed within the context of the present invention.

We claim:

1. An apparatus for the local administration of a bioactive substance and radiation into a prostate comprising:
a plurality of radiation seeds; and
a drug-eluting polymer, wherein said drug-eluting polymer is adapted to release an anti-inflammatory compound.

2. The apparatus of claim 1, further comprising spacers between said radiation seeds.

3. The apparatus of claim 2, wherein said spacers comprise said drug-eluting polymer.

4. The apparatus of claim 1, wherein said radiation seeds are surrounded by said drug-eluting polymer.

5. The apparatus of claim 1, further comprising a sheath surrounding said spacers and said radiations seeds.

6. The apparatus of claim 5, wherein said sheath includes said drug-eluting polymer.

7. The apparatus of claim 1, wherein said anti-inflammatory drug is selected from the group consisting of corticosteroid anti-inflammatory drug, non-steroid anti-inflammatory drug, and novel anti-inflammatory drug.

8. The apparatus of claim 7, wherein said corticosteroid anti-inflammatory drug is selected from the group consisting of corticosterone, cortisone, aldosterone, hydrocortisone acetate, methylprednisolone acetate, triamcinolone acetonide, dexamethasone sodium phosphate, betamethasone sodium phosphate and acetate, budesonide, hydrocortisone, methylprednisilone, prednisolone, prednisone, triamcinilone, alcometasone dipropionate, betamethasone valerate, desoximetasone, fluocinolone, flurandrenolide, fluticasone propionate, hydrocortisone butyrate, hydrocortisone valerate, mometasone furoate, amcinonide, betamethasone dipropionate, diflorasone diacetate, fluocinonide, halcinonide, clobetasol, diflorasone diacetate, halobetasol propionate, fluticasone, beclomethasone, flunisolide, halobetasol propionate, betamethasone valerate, clocortolone pivalate, fluocinolone acetonide, flurandrenolide, prednicarbate, alclometasone dipropionate, and desonide.

9. The apparatus of claim 8, wherein said corticosteroid anti-inflammatory drug is selected from the group consisting of triamcinolone acetonide, methylprednisolone acetate, betamethasone sodium phosphate, and betamethasone acetate.

10. The apparatus of claim 9, wherein said apparatus includes triamcinolone acetonide at a total dose of about 10 milligrams to about 40 milligrams.

11. The apparatus of claim 9, wherein said apparatus includes methylprednisolone acetate at a total dose of about 2 milligrams to about 10 milligrams.

12. The apparatus of claim 9, wherein said apparatus includes betamethasone sodium phosphate at a total dose of about 1 milligram to about 3 milligrams.

13. The apparatus of claim 9, wherein said apparatus includes betamethasone acetate at a total dose of about 1 milligram to about 3 milligrams.

14. The apparatus of claim 7, wherein said non-steroid anti-inflammatory drug is selected from the group consisting of ketoprofen, mefenamic acid, nabumetone, diclofenac, diflunisal, indomethacin, sulindac, flurbiprofen, valdecoxib, celecoxib, rofecoxib, fenoprofen, etodolac, piroxicam, tolmetin, meloxicam, naproxen, oxaprozin, ketorolac, sulindac, phenylbutazone, ibuprofen, floctafenine, and meclofenamate.

15. The apparatus of claim 7, wherein said novel anti-inflammatory drug is selected from the group consisting of minocylcline, colchicine, annexin 1, triptolide, interleukin-4 (IL-4), nuclear factor (NF)-κB inhibitors, licofelone, resvesterol, 5-furoyl-2,2,4-trimethyl-1 ,4-dihydro-1H-1,5-benzodiazepine, N-(substituted)-1-heteroaryl-oxindole-3-carboxamides (wherein the N-substituent is thienyl, furyl, phenyl or substituted phenyl), benzothiazine derivative, that is N-(2-pyridyl)-2-methyl-4-cinnamoyloxy-2H-1,2-benzothiazine-3-carboxamido 1,1-dioxide, 1-heteroaryl-3-acyl-2-oxindoles, substituted 2,3,4,9-tetrahydro-1H-carbazole-1-acetic acid derivatives, 1,5-diaryl pyrazole anti-inflammatory agents, human phosphlipase inhibitory protein (hPIP), substituted 2,3,4,9-tetrahydro-1H-carbazole-1-acetic acid derivatives, 4,5-diaryl-2-(substituted-thio)imidazols and their corresponding sulfoxides and sulfones.

16. The apparatus of claim 1, wherein said apparatus is adapted for use in brachytherapy.

17. The apparatus of claim 1, wherein said apparatus is adapted to be administered to a patient using a Mick applicator.

18. The apparatus of claim 1, wherein said drug-eluting polymer is selected from the group consisting of gums, cellulose ethers, acrylic resins, protein-derived materials, waxes, shellac, hydrogenated castor oil, hydrogenated vegetable oil, alkylcelluloses, acrylic and methacrylic acid polymers and copolymers, cellulose ethers, hydroxyalkylcelluloses, carboxyalkylcelluloses, phosphorylcholine, lactic acid polymer, glycolic acid polymer, polyglycolide, polylactide, poly-ε-caprolactone, poly-dioxanone, and silicon.

19. A method for treating prostate cancer in a patient, comprising:
placing a plurality of radiation seeds into a prostate gland of said patient; and
administering an anti-inflammatory compound into said prostate gland with said radiation seeds.

20. The method of claim 19, wherein said radiation seeds are separated by spacers between said radiation seeds.

21. The method of claim 20, wherein said spacers comprise a drug-eluting polymer.

22. The method of claim 19, wherein said radiation seeds are surrounded by a drug-eluting polymer.

23. The method of claim 20, further comprising a sheath surrounding said spacers and said radiations seeds.

24. The method of claim 23, wherein said sheath includes said drug-eluting polymer.

25. The method of claim 19, wherein said anti-inflammatory drug is selected from the group consisting of corticosteroid anti-inflammatory drug, non-steroid anti-inflammatory drug, and novel anti-inflammatory drug.

26. The method of claim 25, wherein said corticosteroid anti-inflammatory drug is selected from the group consisting of corticosterone, cortisone, aldosterone, hydrocortisone acetate, methylprednisolone acetate, triamcinolone acetonide, dexamethasone sodium phosphate, betamethasone sodium phosphate and acetate, budesonide, hydrocortisone, methylprednisilone, prednisolone, prednisone, triamcinilone, alcometasone dipropionate, betamethasone valerate, desoximetasone, fluocinolone, flurandrenolide, fluticasone propionate, hydrocortisone butyrate, hydrocortisone valerate, mometasone furoate, amcinonide, betamethasone dipropionate, diflorasone diacetate, fluocinonide, halcinonide, clobetasol, diflorasone diacetate, halobetasol propionate, fluticasone, beclomethasone, flunisolide, halobetasol propionate, betamethasone valerate, clocortolone pivalate, fluocinolone acetonide, flurandrenolide, prednicarbate, alclometasone dipropionate, and desonide.

27. The method of claim 26, wherein said corticosteroid anti-inflammatory drug is selected from the group consisting of triamcinolone acetonide, methylprednisolone acetate, betamethasone sodium phosphate, and betamethasone acetate.

28. The method of claim 27, wherein said apparatus includes triamcinolone acetonide at a total dose of about 10 milligrams to about 40 milligrams.

29. The method of claim 27, wherein said apparatus includes methylprednisolone acetate at a total dose of about 2 milligrams to about 10 milligrams.

30. The method of claim 27, wherein said apparatus includes betamethasone sodium phosphate at a total dose of about 1 milligrams to about 3 milligrams.

31. The method of claim 27, wherein said apparatus includes betamethasone acetate at a total dose of about 1 milligrams to about 3 milligrams.

32. The method of claim 25, wherein said non-steroid anti-inflammatory drug is selected from the group consisting of ketoprofen, mefenamic acid, nabumetone, diclofenac, diflunisal, indomethacin, sulindac, flurbiprofen, valdecoxib, celecoxib, rofecoxib, fenoprofen, etodolac, piroxicam, tolmetin, meloxicam, naproxen, oxaprozin, ketorolac, sulindac, phenylbutazone, ibuprofen, floctafenine, and meclofenamate.

33. The method of claim 25, wherein said novel anti-inflammatory drug is selected from the group consisting of minocylcline, colchicine, annexin 1, triptolide, interleukin-4 (IL-4), nuclear factor (NF)-κB inhibitors, licofelone, resvesterol, 5-furoyl-2,2,4-trimethyl-1,4-dihydro-1H-1,5-benzodiazepine, N-(substituted)-1-heteroaryl-oxindole-3-carboxamides (wherein the N-substituent is thienyl, furyl, phenyl or substituted phenyl), benzothiazine derivative, that is N-(2-pyridyl)-2-methyl-4-cinnamoyloxy-2H-1,2-benzothiazine-3-carboxamido 1,1-dioxide, 1-heteroaryl-3-acyl-2-oxindoles, substituted 2,3,4,9-tetrahydro-1H-carbazole-1-acetic acid derivatives, 1,5-diaryl pyrazole anti-inflammatory agents, human phosphlipase inhibitory protein (hPIP), substituted 2,3,4,9-tetrahydro-1H-carbazole-1-acetic acid derivatives, 4,5-diaryl-2-(substituted-thio)imidazols and their corresponding sulfoxides and sulfones.

34. The method of claim 19, wherein said apparatus is adapted for use in brachytherapy.

35. The method of claim 19, wherein said placing step is accomplished using a Mick applicator.

36. The method of claim 19, wherein said placing step and said administering step are accomplished using a Mick applicator.

37. The method of claim 21, wherein said drug-eluting polymer is selected from the group consisting of gums, cellulose ethers, acrylic resins, protein-derived materials, waxes, shellac, hydrogenated castor oil, hydrogenated vegetable oil, alkylcelluloses, acrylic and methacrylic acid polymers and copolymers, phosphorylcholine, cellulose ethers, hydroxyalkylcelluloses, and carboxyalkylcelluloses, lactic acid polymer, glycolic acid polymer, polyglycolide, polylactide, poly-ε-caprolactone, poly-dioxanone, and silicon.

38. A method of treating prostate cancer in a patient, comprising:
exposing said prostate gland of said patient to radiation; and
administering an anti-inflammatory drug into said prostate gland.

39. The method of claim 38, wherein said exposing step comprises exposing said prostate to a radiation beam.

40. The method of claim 38, wherein said exposing step comprises inserting radiation seeds into said prostate.

41. The method of claim 38, wherein said administering step comprises inserting a drug-eluting polymer into said patient.

42. The method of claim 41, wherein said drug-eluting polymer includes said anti-inflammatory drug.

43. The method of claim 42, wherein said anti-inflammatory drug is selected from the group consisting of corticosteroid anti-inflammatory drug, non-steroid anti-inflammatory drug, and novel anti-inflammatory drug.

44. The method of claim 38, wherein said administering step includes inserting a depot formulation into said patient.

45. The method of claim 40, wherein said radiation seeds are coated with said anti-inflammatory drug.

46. An apparatus for the local administration of a bioactive substance and radiation into a prostate comprising:
a plurality of radiation seeds; and
a drug-eluting substance, wherein said drug-eluting substance is adapted to release a chemotherapeutic compound.

47. The apparatus of claim 46, wherein said drug-eluting substance is selected from the group consisting of drug-eluting polymer or ceramic compound.

48. A method of treating inflammation of a prostate gland in a patient, comprising the step of:
administering a composition directly into said prostate gland, said composition comprising:
a drug-eluting polymer or ceramic compound; and
an anti-inflammatory agent.

49. The method of claim 48, wherein said anti-inflammatory agent is selected from the group consisting of corticosteroid anti-inflammatory drug, non-steroid anti-inflammatory drug, and novel anti-inflammatory drug.

50. The method of claim 49, wherein said corticosteroid anti-inflammatory drug is selected from the group consisting of corticosterone, cortisone, aldosterone, hydrocortisone acetate, methylprednisolone acetate, triamcinolone acetonide, dexamethasone sodium phosphate, betamethasone sodium phosphate and acetate, budesonide, hydrocortisone, methylprednisilone, prednisolone, prednisone, triamcinilone, alcometasone dipropionate, betamethasone valerate, desoximetasone, fluocinolone, flurandrenolide, fluticasone propionate, hydrocortisone butyrate, hydrocortisone valerate, mometasone furoate, amcinonide, betamethasone dipropionate, diflorasone diacetate, fluocinonide, halcinonide, clobetasol, diflorasone diacetate, halobetasol propionate, fluticasone, beclomethasone, flunisolide, halobetasol propionate, betamethasone valerate, clocortolone pivalate, fluocinolone acetonide, flurandrenolide, prednicarbate, alclometasone dipropionate, and desonide.

51. The method of claim 50, wherein said corticosteroid anti-inflammatory drug is selected from the group consisting of triamcinolone acetonide, methylprednisolone acetate, betamethasone sodium phosphate, and betamethasone acetate.

52. The method of claim 51, wherein said apparatus includes triamcinolone acetonide at a total dose of about 10 milligrams to about 40 milligrams.

53. The method of claim 51, wherein said apparatus includes methylprednisolone acetate at a total dose of about 2 milligrams to about 10 milligrams.

54. The method of claim 51, wherein said apparatus includes betamethasone sodium phosphate at a total dose of about 1 milligram to about 3 milligrams.

55. The method of claim 51, wherein said apparatus includes betamethasone acetate at a total dose of about 1 milligram to about 3 milligrams.

56. The method of claim 49, wherein said non-steroid anti-inflammatory drug is selected from the group consisting of ketoprofen, mefenamic acid, nabumetone, diclofenac, diflunisal, indomethacin, sulindac, flurbiprofen, valdecoxib, celecoxib, rofecoxib, fenoprofen, etodolac, piroxicam, tolmetin, meloxicam, naproxen, oxaprozin, ketorolac, sulindac, phenylbutazone, ibuprofen, floctafenine, and meclofenamate.

57. The method of claim 49, wherein said novel anti-inflammatory drug is selected from the group consisting of minocylcline, colchicine, annexin 1, triptolide, interleukin-4 (IL-4), nuclear factor (NF)-κB inhibitors, licofelone, resvesterol, 5-furoyl-2,2,4-trimethyl-1,4-dihydro-1H-1,5-benzodiazepine, N-(substituted)-1-heteroaryl-oxindole-3-carboxamides (wherein the N-substituent is thienyl, furyl, phenyl or substituted phenyl), benzothiazine derivative, that is N-(2-pyridyl)-2-methyl-4-cinnamoyloxy-2H-1,2-benzothiazine-3-carboxamido 1,1-dioxide, 1-heteroaryl-3-acyl-2-oxindoles, substituted 2,3,4,9-tetrahydro-1H-carbazole-1-acetic acid derivatives, 1,5-diaryl pyrazole anti-inflammatory agents, human phosphlipase inhibitory protein (hPIP), substituted 2,3,4,9-tetrahydro-1H-carbazole-1-acetic acid derivatives, 4,5-diaryl-2-(substituted-thio)imidazols and their corresponding sulfoxides and sulfones.

58. The method of claim 48, wherein said administering step occurs following brachytherapy.

59. The method of claim 48, wherein said administering step occurs following a biopsy of said prostate gland.

60. The method of claim 48, wherein said administering step occurs following exposure of said prostate gland to radiation.

61. The method of claim 48, wherein said drug-eluting polymer is selected from the group consisting of gums, cellulose ethers, acrylic resins, protein-derived materials, waxes, shellac, hydrogenated castor oil, hydrogenated vegetable oil, alkylcelluloses, acrylic and methacrylic acid polymers and copolymers, cellulose ethers, hydroxyalkylcelluloses, carboxyalkylcelluloses, phosphorylcholine, lactic acid polymer, glycolic acid polymer, polyglycolide, polylactide, poly-ε-caprolactone, poly-dioxanone, and silicon.
